(19) United States
(12) Patent Application Publication
(10) Pub. No.: US 2009/0241947 A1
(43) Pub. Date: Oct. 1, 2009

(54) APPARATUS FOR CONTROLLED AND AUTOMATIC MEDICAL GAS DISPENSING

(75) Inventors: Remo Bedini, Pisa (IT); Alessandro Navari, Pietrasanta (IT); Andrea Belardinelli, Firenze (IT); Graziano Palagi, Pontasserchio (IT); Bruno Formichi, Navacchio (IT)

Correspondence Address: DENNISON, SCHULTZ & MACDONALD, 1727 KING STREET, SUITE 105, ALEXANDRIA, VA, 22314, US

(73) Assignee: CNR- CONSIGLIO NAZIONALE DELLE RICHERCHE, Roma (IT)

(21) Appl. No.: 12/307,650

(22) PCT Filed: Jul. 19, 2007
(86) PCT No.: PCT/IB07/02040

§ 371 Date: Jan. 23, 2009

(30) Foreign Application Priority Data
Jul. 20, 2006 (IT) PI2006A000090
Jul. 20, 2006 (IT) PI2006A000091

(51) Int. Cl. A61M 16/12; A61B 5/08

(52) U.S. Cl. 128/203.14; 600/537

(57) ABSTRACT

Apparatus for supplying in a controlled and automatic way boli of nitric oxide (NO) to patients ‘(50)’ affected by respiratory diseases. The apparatus comprises a reservoir (20) connected an electro-valve (21) for adjusting the flow. The electro-valve (21) is switched on/off by a microprocessor (40) in order to supply the medical gas contained in the reservoir, (20) in synchronism with the respiratory rhythm of the patient (50). This rhythm can be outlined on the basis of the temperature values of the respiration flow by measuring, means (80) comprising a first thermistor (81) and a second thermistor (82) electrically connected to each other. The temperature values are then computed by the micropocessor (40). The flow controlled is sent to the respiratory airways of the patient (50) through a thin nasal tube (2).

### FIELD OF THE INVENTION

The present invention relates to the medical field and more precisely it relates to an apparatus for supplying in a controlled and automatic way a determined amount of medical gas to patients to which it is useful reducing the pulmonary resistance to decrease the pulmonary pressure and/or to increase the heart range.

### BACKGROUND OF THE INVENTION

As well known, the supply of controlled gas for therapeutic purposes is now a widespread clinical practice, in special way in oxygen therapy for treating diseases such as chronic obstructive bronchopneumopathy (BPCO).

Furthermore, alternative therapies have been studied that provide the supply of other gas, for example nitrogen monoxide (NO), also called nitric oxide, for diagnosing and treating diseases such as primitive pulmonary hypertension.

In particular, it has been found that nitric oxide is capable of inducing vascular muscle release. Furthermore, nitric oxide has also a high rapidity of action, a short half life and does not bring about phenomena of tachyphylaxis, i.e. a rapidly decreasing response to a drug. Nitric oxide is an effective drug if inhaled; in fact if it is administered in this form it produces dilation exclusively on the pulmonary vessels involved in gaseous exchanges, improving then the ventilation/perfusion ratio (V/Q), and avoiding detrimental arterial-venous “shunts”.

However, the use of nitric oxide as medical gas is not widespread yet, since the existing devices are not capable of supplying this medical gas in a desired way. More precisely, the prior art devices are not capable of supplying nitric oxide at low dosage (5-40 ppm) and to limit the time of contact between the inhaled gases, that the patient must breath in, and nitric oxide. This condition is, in particular, essential since it aims at avoiding the combination of nitric oxide with oxygen and then the production of nitrogen dioxide (NO₂), which is a gas toxic by inhalation. The latter can react in turn with the water, forming nitric acid (HNO₃) that is a particularly reactive and then dangerous acid.

The prior art systems provide, in particular, the use of pulmonary ventilators for delivering the drug to the patients. This solution causes a significant production of noxious compounds for large volumes of nitric oxide (NO) remaining a long time in contact with oxygen (O₂). The gas is fed when breathing spontaneously, but with a continuous delivery, whereby the gas supplied when breathing out is dispersed in the environment, where indeed a big amount of NO can react with oxygen creating the dangerous nitrogen dioxide (NO₂). These applications cause a significant environmental pollution.

Among the known systems used for supplying medical gas, there are some of them that provide devices for measuring the respiratory phases in order to selectively adjust the supply of the gas, in particular oxygen, in patients affected by respiratory insufficiency. The devices known for measuring the respiratory phases provide the use of sensors of many kinds.

For example, sensors used to this object are hot wire thermo-anemometers. They measure a fluid speed by measuring the amount of heat exchanged by convection with a fluid that laps it. The heat dissipated by the hot wire invested by the fluid flow depends on different factors among which the temperature of the wire, its geometry, the temperature and the speed of the fluid. In particular, the temperature of the wire can be calculated by measuring an electric resistance.

However, since the speed of breathing in and out changes in a narrow range of values, between 0 and about 20 litres/sec., the resistance variation of the wire during the operation of the sensor is very low. Therefore, it is necessary to carry out a measurement of the variation of resistance of the wire with high precision in order to calculate the speed of the fluid. Furthermore, the sensors of this type do not provide a high speed of response, and then their use is limited to determined applications such as the supply of oxygen, for which it is not necessary to supply the gas in perfect synchronism with the respiratory rhythm of the patient.

### SUMMARY OF THE INVENTION

It is then an feature of the present invention to provide an apparatus for supplying in a controlled and automatic way a determined amount of medical gas to a patient, which overcomes the disadvantages of the prior art.

It is another feature of the present invention to provide such an apparatus for supplying the medical gas in synchronism with the respiratory rhythm of the patient.

It is also an feature of the present invention to provide such an apparatus that has a minimum encumbrance and that can be easily used by patients, both in hospitals and at home, in conditions of maximum safety.

It is a particular feature of the present invention to provide an apparatus for supplying in a controlled and automatic way nitric oxide, which assures a minimum contact between nitric oxide and oxygen and that then can avoid the production of nitrogen dioxide and nitric acid.

It is a particular feature of the present invention to provide a sensor for the detection of the respiratory phases of a patient, adapted to overcome the disadvantages of the similar apparatus of the prior art.

It is another particular feature of the present invention to provide a sensor for the detection of the respiratory phases of a patient capable of assuring a high speed of response.

It is to further particular feature of the present invention to provide a sensor for the detection of the respiratory phases of a patient that is structurally easy and not expensive to make with respect to the sensors of the prior art.

These and other features are accomplished with one exemplary apparatus for supplying to a patient in a controlled and automatic way a determined amount of at least one medical gas, in particular, nitric oxide and/or oxygen, comprising:
means for generating at least one flow of medical gas,
means for adjusting the, or each, flow of medical gas,
means for connecting the, or each, flow of medical gas with the respiratory airways of the patient,
means for measuring the respiratory rhythm of the patient;
means for operating said means for adjusting so that said supply of the, or each, flow of medical gas occurs in synchronism with the respiratory phases of the patient;

whose main feature is that said means for measuring the respiratory rhythm comprises at least one thermistor, in pneumatic connection with the respiratory airways of the patient, adapted to measure a temperature value and to transmit it to means for correlating it to the inspiratory phase or to the expiratory phase of the patient.

Preferably, the means for measuring the respiratory rhythm comprises:
a first thermistor in pneumatic connection with the respiratory airways of the patient,
a second thermistor arranged in an environment at a reference temperature, said first and second thermistors being in electric connection with each other;
means for analysing the temperature values measured by said first and second thermistors and to provide a differential signal;
means for correlating said differential signal to the inspiratory phase or to the expiratory phase of the patient.

More in detail, the first thermistor is not in direct contact with the respiratory airways of the patient, but is in any case in a lap contact with the breathed air flow. This avoids both a pollution of the means for measuring by the patient's exhaled flow, and a possibility of having induced currents discharged from the means for measuring towards the patient.

In particular, the means for correlating are adapted to calculate a derivative of the differential signal and to compare its value with a threshold value. If the value of the derivative is less than the threshold value the means for correlating associate to it the breathing in phase. If, instead, the value of the derivative is larger than the threshold value, the means for correlating associate to it the expiratory phase of the patient.

In addition, or alternatively, to the thermistor the means for measuring the respiratory rhythm of the patient can comprise:
a sensor having a suitable rapidity for measuring the speed of the inspired flow of the patient,
a sensor having a suitable rapidity for measuring the speed of the expired flow of the patient.

In this case, means are provided for correlating the speed differential measure, made by the first and the second sensor, with the respiratory rhythm of the patient.

In an exemplary embodiment of the invention, the means for measuring the respiratory rhythm, comprises a first thermospeedometric sensor and a second thermospeedometric sensor electrically connected to each other, said first sensor being in pneumatic connection with said means for connecting said flow and said second sensor being in communication with the environment at a reference temperature.

In a practical embodiment, the step of the detection of the respiratory rhythm of the patient is carried out by measuring instantly the temperature difference between the air breathed in flow/out by the patient and the environment. In general, indeed, the temperature of the breathed in flow is less than the breathed out flow and through the use of specific algorithms starting from a differential temperature measure it is possible to define a chart of the respiratory flow of the patient responsive to time. A borderline case can occur if the temperature of the environment is higher than the breathed out flow. In this case the sensor detects a signal in opposite phase with respect to the respiratory phases. In the case, instead, where the breathed in air and the breathed out flow have the same temperature, the detection of the respiratory rhythm is made through the measurement of the speed. In fact, the speed of the breathed in flow is much greater than the speed of the breathed out flow. Still another possibility is measuring the humidity of the two flows, since the humidity of the breathed flow is much greater in expiration than in inspiration.

Preferably, the means for measuring the respiratory rhythm of the patient comprises a first and a second semiconductor diode in direct polarization. In particular, the use of direct polarization semiconductor diodes ensures a high speed of response, in particular greater than other types of thermistors, and allows an extremely simple circuit architecture. In detail, the sensor exploits the fact that in a p-n polarized junction, such as that of a semiconductor diode, for temperatures T>30 K the direct voltage V_{f} is responsive about linearly to the temperature, in case of fixed current I_{f}, as expressed by the equation: V_{f}=Vₒ−g(I_{f})·T. Wherein slope g(I_{f}) depends only slightly on the polarization current.

In particular, the first diode is arranged according to a duct having a measured cross section whereby it is possible to calculate the flow of the breath of the patient by said temperature values. This to avoid electric shock towards the patient during the monitoring step.

In particular, the duct has one end in the airways of the patient and the other end external to them at which is located said first diode.

Advantageously, furthermore, means are provided for measuring at least one variable operative value responsive to the, or to each, gas flow, said means for measuring being selected from the group comprised of:
at least one temperature sensor,
at least one pressure sensor,
at least one flow rate sensor,

or a combination thereof.

Furthermore, means can be provided for monitoring the presence of pollutants in the environment around the patient; in particular in case of supplying nitric oxide the concentration of nitrogen dioxide (NO₂) present in the environment can be determined. The means for monitoring the presence of pollutants can be, in particular, associated with visual and/or sonic alarm that is activated when a predetermined threshold value is exceeded.

Advantageously, means are also provided for measuring at least one physiological parameter of the patient selected from the group comprised of:
arterial oxygen saturation (SpO₂),
arterial partial pressure of carbon dioxide (PaCO₂),
a combination thereof.

The apparatus as above described, allows to supply nitric oxide boli, for diagnostic and/or therapeutic purposes, for example in patients affected by BPCO. Nitric oxide, in fact, has to be given in boli for the duration of a few ms, to minimize its contact with oxygen with which it reacts creating toxic compounds such as nitrogen dioxide and, in the presence of humidity, also acid substances. Therefore, the possible supply of oxygen, where it is necessary for patients treated with nitric oxide, has to be done for each respiratory cycle only after that nitric oxide has been supplied.

Advantageously, the means for analysing send the data to a remote central unit at which a specialist can work to examine immediately the data and to intervene in case of need. In particular, the data can also be sent to a server and left accessible to a doctor in a second time.

According to particular aspect of the invention, an apparatus for measuring the respiratory phases of a patient comprises:
a first element responsive to temperature in pneumatic connection with the respiratory airways of the subject,
a second element responsive to temperature arranged in an environment at a reference temperature, said first and second element responsive to temperature being in electric connection with each other;
means for analysing the temperature values measured by said first and second elements and to provide a differential signal;
means for correlating the differential signal to the inspiratory phase or to the expiratory phase of the subject;

whose main feature is that each said first and second element responsive to temperature comprise a diode. In particular, the first and second diode are semiconductor diodes with direct polarization.

Preferably, the first and the second diode form a thermospeedometric sensor.

Advantageously, the first element responsive to temperature is arranged according to a duct having a measured cross section whereby it is possible to calculate the flow of the breath of the patient by the data relative to temperature.

In particular, the first diode is arranged according to a duct that in use has one end in the airways of the patient and the other end external to them at which is located the diode same.

### BRIEF DESCRIPTION OF THE DRAWINGS

The invention will be made clearer with the following description of an exemplary embodiment thereof, exemplifying but not limitative, with reference to the attached drawings wherein:

FIG. 1 diagrammatically shows an apparatus for controlled and automatic supply of a medical gas to a patient, according to the present invention;

FIG. 2 shows in detail a sensor that can be used in the apparatus of FIG. 1 in operative conditions for highlighting some functional aspects,

FIG. 3 shows diagrammatically a chart relative to the course versus time of the respiratory flow of a patient,

FIG. 4 shows diagrammatically an alternative exemplary embodiment of the apparatus of FIG. 1;

FIG. 5 shows in a longitudinal cross section a thin tube in which is used in the sensor of FIG. 2;

FIG. 6 shows a detail a thin nasal tube in which an element of the sensor of FIG. 2 is inserted;

FIG. 7 shows diagrammatically a block diagram of various operations through which the respiratory phases of a patient are detected by the means for measuring the respiratory phases of the invention.

### DESCRIPTION OF PREFERRED EXEMPLARY EMBODIMENTS

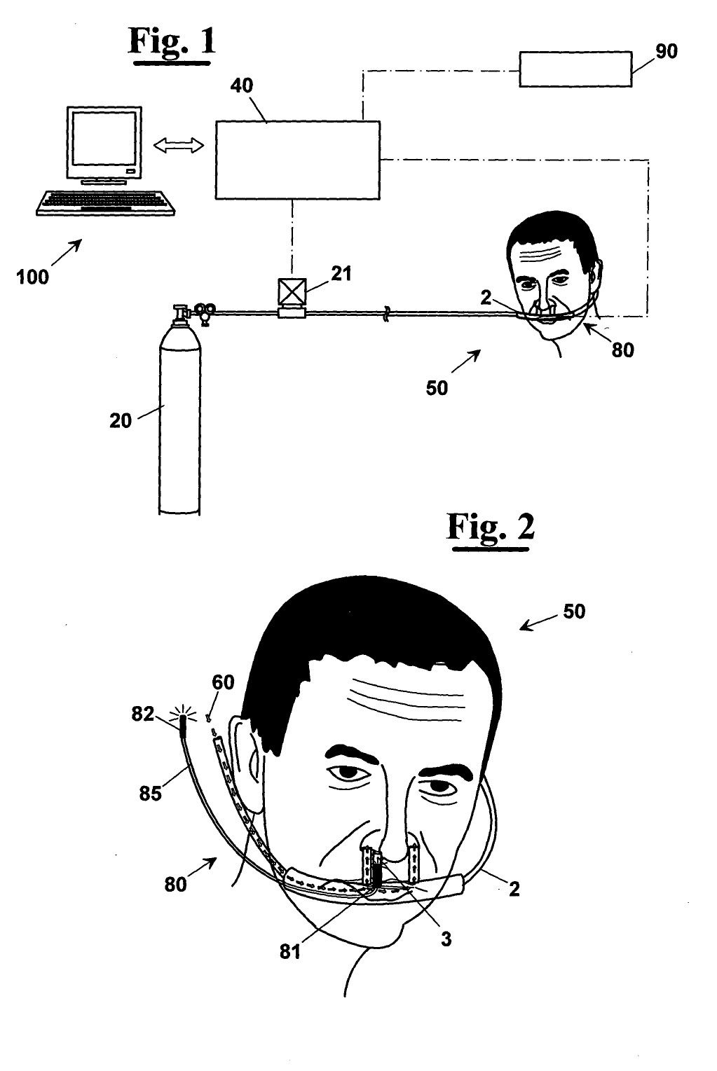

As diagrammatically shown in FIG. 1, the present invention relates to an apparatus 1 for supplying boli of a medical gas, in particular nitric oxide, in a controlled and automatic way, to a patient 50 affected by respiratory diseases. Apparatus 1 comprises, in particular, means for generating at least one flow of nitric oxide, such as a pressurized reservoir 20 connected to means for supplying in a controlled way the gas flow, for example an electro-valve 21. In particular, electro-valve 21 is switched by a microprocessor 40 on the basis of the course of the respiratory rhythm of patient 50.

More in detail, the respiratory rhythm is derived on the basis of a temperature value detected by a thermistor and computed by microprocessor 40. A controlled flow 60 thus generated can be released in the respiratory airways of patient 50 through a thin nasal tube 2 (FIG. 2).

The gas supply to patient 50 is, then, made under a “feedback” on the respiratory rhythm. This allows supplying the boli of nitric oxide (NO) in synchronism with the respiratory rhythm of patient 50, i.e. at a maximum breathing in depression corresponding to a maximum pulmonary vasodilation. This way, nitric oxide is completely adsorbed by the body, and then a pollution of NO and/or NO₂ in the breathed out air flow is practically zero.

As diagrammatically shown in FIG. 3, the respiratory rhythm begins with an inspiration phase, portion 200 of the chart, comprising a starting inspiration phase during which there is a maximum inspiratory muscle compliance followed by a step of inspiratory latency, portion 201. At the end of the inspiration phase there is an expiration phase, portion 202 in the chart of FIG. 3, comprising the expiratory phase, where the pulmonary gas is expelled, and a following expiratory latency, portion 203.

For supplying nitric oxide (NO), in synchronism with the respiratory rhythm of the patient it is therefore necessary to know in real time the beginning and the end of each respiratory phase, in order to switch instantly the opening/closing position of electro-valve 21. This can be obtained measuring instantly the temperature difference existing between the breathed in/out flow by patient 50 and the environment.

For example, the temperature difference between the breathed out flow and the breathed in flow can be determined by a temperature sensor 80 shown in FIG. 2. In particular, sensor 80 comprises a first diode 81 and a second diode 82 electrically connected by means of a wire 85. In a preferred exemplary embodiment, diodes 81 and 82 are semiconductor diodes in direct polarization.

In particular, diode 81 is pneumatically connected to a duct, for example to thin nasal tube 2, where the respiratory flow of patient 50 passes. More in detail, diode 81 is arranged in a branch 93 of thin nasal tube 2 that in use has one end arranged in the respiratory airways of patient 50 and the other end external to them. The end of branch 93 in the respiratory airways allows conveying the air to lap diode 81. Diode 82 is arranged at a distance from diode 81 and is arranged in the environment at a reference temperature T_{amb}.

As shown in detail in FIG. 6, on a side surface of branch 93, side openings 95 can be made so that even if a main opening 94 is blocked, the flows of inspired and expired air of patient 50 reach in any case diode 81. This arrangement is provided to avoid electric shock to patient 50, without however affecting the precision of detecting the temperature of the air by sensor 80. This way, in fact, diode 81 is in a lap contact with the air flow of patient 50 without the risk contacting with the nasal mucosa.

The solution above described has a very high speed of response and then allows outlining instantly the course of the respiratory rhythm of patient 50. By the temperature value obtained from sensor 80 it is possible, in fact, to calculate the course of the respiratory rhythm of patient 50, for example by a microprocessor 40, and by means 100 for correlating the differential signal between the inspiratory phase or the expiratory phase of the patient (FIG. 1). In particular, microprocessor 40 can be put in connection with a remote server at which for example a specialist operates ready to intervene in case of need.

The main steps of the means for measuring the respiratory phases of patient 50 are diagrammatically shown in a block diagram 150 of FIG. 7. Starting from the temperature values measured by diodes 81 and 82 (blocks 151 and 152) a differential signal is generated by means of a resistance bridge (block 153). The differential signal product is then amplified (block 154) and then correlated to the different respiratory phases of the patient (block 155).

In particular, the signal generated by the resistance bridge is sinusoidal, i.e. increases when breathing out and decreases during when breathing in. The definition and then the discrimination between the increasing and decreasing portions is made calculating the derivative of the differential signal by means of an operational amplifier (block 156). Preferably, the operational amplifier has a very low time constant so that it has a high speed of response. The portions having a positive derivative, i.e. the increasing portions, are associated with the expiratory phase, whereas the portions having a negative derivative are associated with the inspiratory phase of the patient. This way, the whole course of the respiratory phases of the patient is instantly outlined (block 157).

For compensating possible errors of detection due to noise it is possible to set a threshold value, for example equal to −1, as discriminating reference for the derivative, for distinguishing the increasing and the decreasing portions.

What above described is possible since the temperature of the breathed in flow is normally less than the temperature of the breathed out flow, whereby the differential measure of such temperature allows, using specific algorithms, to determine the respiratory rhythm.

A borderline case can occur if the temperature of the environment is higher than that of the breathed out flow. In this case, sensor 80 detects a signal in opposite phase with respect to the respiratory cycle. In the case, instead, where the breathed in air and the breathed out flow have the same temperature, the detection of the respiratory rhythm can be made through the measurement of the flow speed. In fact, the speed of the breathed in flow is much greater than the speed of the breathed out flow. In this case, then, sensor 80 can be a thermospeedometer. Still another possibility is measuring the humidity of the two flows, since the humidity of the breathed out flow is much greater in expiration that in inspiration. When breathing out the thin nasal tube 2 is in fact crossed by a flow of warm and humid air, whereas during the inspiratory phase it is crossed by cold air having a lower humidity.

The data of temperature, speed and humidity relative to the air flow inspired and exhaled by patient 50 are then computed by microprocessor 40 and converted on values relative to the respiratory rhythm.

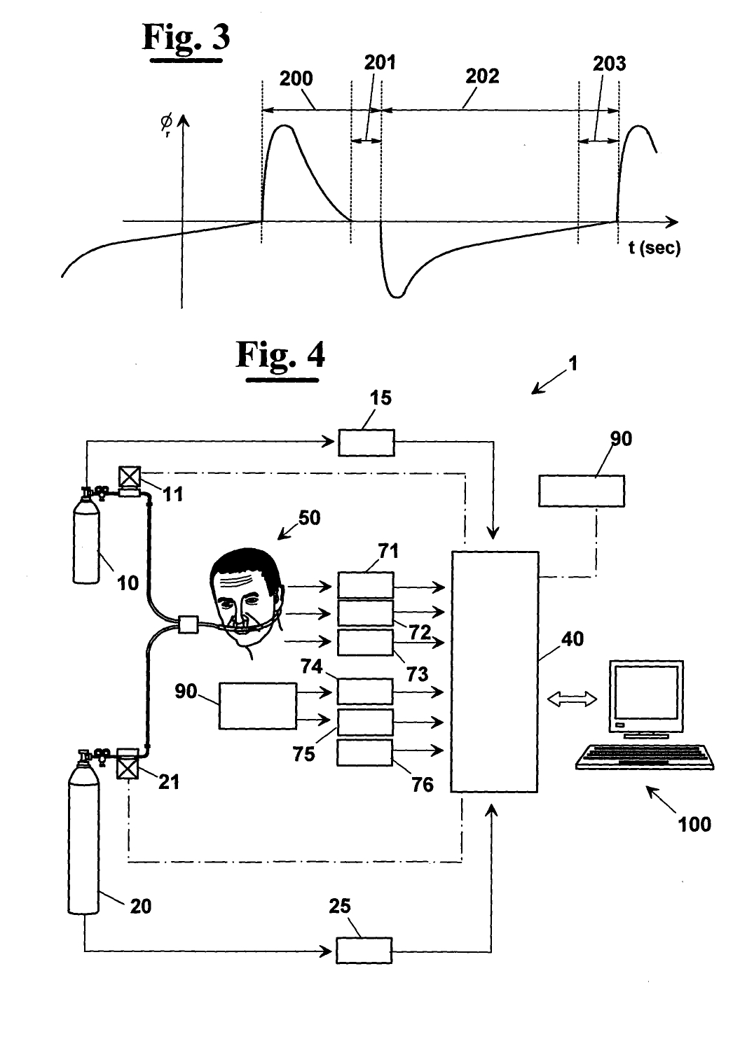

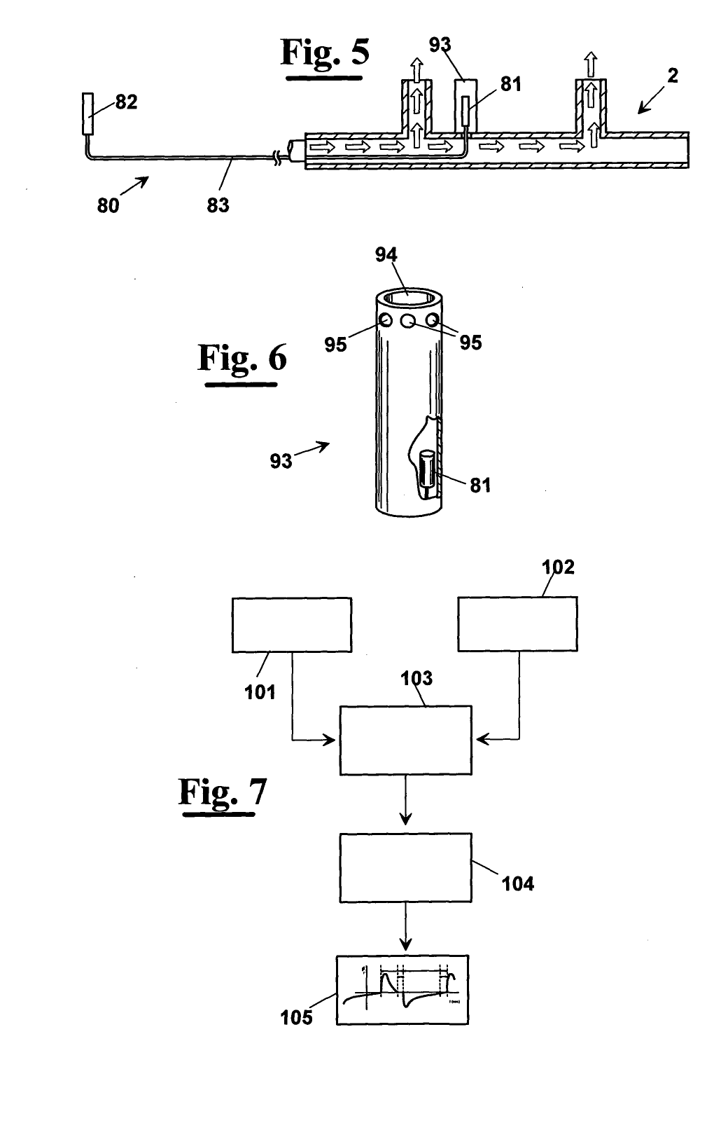

The apparatus 1 can comprise, furthermore, means for measuring at least one monitored physiological parameter. In particular, the sensor used for measuring the physiological parameter, changes according to the administered medical gas. For example, In the case shown in FIG. 4, where there is a combined supply of oxygen (O₂), drawn by a reservoir 10, and of nitric oxide (NO), drawn by a reservoir 20, the physiological parameters of patient 50 can be: arterial oxygen saturation (SpO₂), arterial partial pressure of carbon dioxide (PaCO₂) and respiratory rhythm, respectively blocks 71, 72 and 73 of FIG. 4. The sensors used for measuring such physiological parameters can work, for example, exploiting the technique of transcutaneous measure. This technique exploits the phenomenon of the blood gases, oxygen and carbon dioxide, conveyed through the tissues of the body and of the skin that allows a measurement by means of a surface sensor. The partial pressures of oxygen and carbon dioxide determined at the skin surface are correlated with their hematic levels that can then be determined with high precision. Alternatively, it is possible to use chemical sensors, such as a capnograph for carbon dioxide, which allows a measurement of exhaled CO₂ (EtCO₂) and then of the hematic CO₂ that can be correlated to it.

Apparatus 1 can, furthermore, provide means 90 for monitoring a pollution of the environment around patient 50, capable of measuring the concentration of NO₂ in it present, block 74. In case of exceeding a predefined threshold value the means 90 emit an alarm signal of visual and/or audio type. Sensors can be, furthermore, provided for measuring the room temperature, block 75. Other measuring instruments that can be provided can be pressure sensors on the lines of oxygen and of nitric oxide, block 15 and block 25, respectively, which can be flow sensors, not shown in the figure.

The apparatus 1, according to the above described invention, is capable of providing a valid technological aid for decentralizing the assistance towards the home of the patient, also jointly with portable systems for oxygen therapy, that can also be used in therapy on self-moving patients.

The foregoing description of a specific embodiment will so fully reveal the invention according to the conceptual point of view, so that others, by applying current knowledge, will be able to modify and/or adapt for various applications such an embodiment without further research and without parting from the invention, and it is therefore to be understood that such adaptations and modifications will have to be considered as equivalent to the specific embodiment. The means and the materials to realise the different functions described herein could have a different nature without, for this reason, departing from the field of the invention. It is to be understood that the phraseology or terminology employed herein is for the purpose of description and not of limitation.

1. Apparatus for supplying in a controlled and automatic way a determined amount of at least one medical gas to a patient comprising:
means for generating at least one flow of medical gas,
means for adjusting said, or each, flow of medical gas,
means for connecting said, or each, flow of medical gas with the respiratory airways of said patient,
means for measuring the respiratory rhythm of the patient;
means for operating said means for adjusting so that said supply of the, or each, flow of medical gas occurs in synchronism with the respiratory phases of the patient;
characterised in that said means for measuring said respiratory rhythm comprises at least one thermistor in pneumatic connection with the respiratory airways of the patient for measuring a temperature value and transmit it to means for correlating the measured temperature value to the inspiratory phase or to the expiratory phase of the patient.

2. Apparatus, according to claim 1, wherein, furthermore, means are provided for measuring at least one variable operative value relative to said, or to each, gas flow, said means for measuring being selected from the group comprised of:
at least one temperature sensor,
at least one pressure sensor,
at least one flow rate sensor,
or a combination thereof.

3. Apparatus, according to claim 1, wherein, furthermore, means are provided for monitoring the presence of pollutants in the environment around said patient, said means for monitoring being associated with visual and/or sonic alarm that is activated when a, predetermined threshold value is exceeded.

4. Apparatus, according to claim 4, wherein said means for monitoring the presence of pollutants in the environment around said patient provide at least a sensor measuring the concentration of nitrogen dioxide (NO₂).

5. Apparatus, according to claim 1, wherein, furthermore, means are provided for measuring at least one physiological parameter selected from the group comprised of:
arterial oxygen saturation (SpO₂),
arterial partial pressure of carbon dioxide (PaCO₂),
a combination thereof.

6. Apparatus, according to claim 1, wherein said means for measuring the respiratory rhythm comprises:
a first thermistor in pneumatic connection with the respiratory airways of the patient,
a second thermistor arranged in an environment at a reference temperature, said first and second thermistors being in electric connection with each other;
means for analysing the temperature values measured by said first and second thermistors and to provide a differential signal;
means for correlating said differential signal to the inspiratory phase or to the expiratory phase of said patient.

7. Apparatus, according to claim 6, wherein said means for correlating are adapted to calculate the derivative of said differential signal and to compare it with a threshold value, said means for correlating associating values of the derivative less than said threshold value to the inspiratory phase and values of the derivative higher than said threshold value to the expiratory phase of said patient.

8. Apparatus, according to claim 1, wherein said means for measuring said respiratory rhythm comprises, furthermore:
a first sensor having a suitable rapidity for measuring the speed of the inspired flow of the patient,
a second sensor having a suitable rapidity for measuring the speed of the expired flow of the patient,
means for correlating a speed differential measure made by the first and the second sensor with the respiratory rhythm of the patient.

9. Apparatus, according to claim 1, wherein said means for measuring said respiratory rhythm provide a first thermospeedometric sensor and a second thermospeedometric sensor electrically connected, said first sensor being in pneumatic connection with said means for connecting said flow and said second sensor being in communication with the environment at a reference temperature.

10. Apparatus, according to claim 6, wherein said first and said second thermistors are diodes.

11. Apparatus, according to claim 10, wherein said first and said second diodes are silicon semiconductor diodes in direct polarization.

12. Apparatus, according to claim 6, wherein said first thermistor is associated to a duct having a measured cross section whereby it is possible to calculate the flow of the breath of the patient by said temperature values.

13. Apparatus, according to claim 12, wherein said duct has one end in the airways of the patient and the other end external to them at which said diode is located.

14. Apparatus, according to claim 5, wherein a remote control unit is provided, in particular an internet server, and wherein said means for analysing send the data relative to said physiological parameter to said remote control unit.

15. Apparatus for measuring the respiratory phases of a patient, comprising:
a first element responsive to temperature in pneumatic connection with the respiratory airways of the subject,
a second element responsive to temperature arranged in an environment at a reference temperature, said first and second element responsive to temperature being in electric connection with each other;
means for analysing the temperature values measured by said first and second element and to provide a differential signal;
means for correlating said differential signal to the inspiratory phase or to the expiratory phase of the subject;
characterised in that said first and second elements responsive to temperature are diodes.

16. Apparatus for measuring the respiratory phases of a patient, according to claim 15, wherein said diodes are silicon semiconductor diodes in direct polarization.
